(19) United States
(12) Patent Application Publication
(10) Pub. No.: US 2024/0398676 A1
(43) Pub. Date: Dec. 5, 2024

(54) COSMETIC METHOD FOR TREATING HAIR WITH OZONE

(71) Applicant: Federico Montanari, Argelato BO (IT)

(72) Inventors: Federico Montanari, Argelato BO (IT); Flavio Venturi, Faenza RA (IT); Flavio Garoia, Bologna BO (IT); Davide Marchesini, Pieve di Cento BO (IT)

(73) Assignee: Federico Montanari, Argelato BO (IT)

(21) Appl. No.: 18/689,987

(22) PCT Filed: Sep. 1, 2022
(86) PCT No.: PCT/IB2022/058220

§ 371 Date: Mar. 7, 2024

(30) Foreign Application Priority Data
Sep. 8, 2021 (IT) 102021000023231

(51) Int. Cl. A61K 8/22; A45D 7/04; A45D 19/00; A61Q 5/00

(52) U.S. Cl. CPC A61K 8/22; A45D 7/04; A45D 19/0066; A61Q 5/00

(57) ABSTRACT

The object of the invention relates to a cosmetic method for hair care comprising the following steps: (a) conducing a technical treatment on the hair, comprising at least the steps of applying on the hair one or more compositions functional to the technical treatment; leaving the one or more compositions functional to the technical treatment in place on the hair; washing the hair to remove from the hair the one or more compositions functional to the technical treatment; (b) applying ozone on the hair for a temporal period comprised between 3 and 20 minutes and at a dosage comprised between 180 mg/h and 400 mg/h, wherein the ozone application step (b) is carried out on damp hair, at the end of the step of conducing of the technical treatment (a).

### FIELD OF THE INVENTION

The field of the present invention concerns an ozone-based cosmetic method for hair care.

### BACKGROUND ART

Ozone therapy is an ozone-based therapy or treatment that is known to produce a number of benefits that can counteract a wide range of diseases (disc herniation, acne, phlebitis, fibromyalgia, infections, haemorrhoids, osteoarthritis, cellulite, ulcers, burns, osteoporosis). This treatment makes it possible to significantly improve the body's defence systems against the action of free radicals, which are the main responsible for cellular ageing and tissue oxidation.

Ozone O₃ is a gas soluble in aqueous solution (˜13 times more than O₂ at 0-30° C.) with a solubility inversely proportional to temperature and pH. It is also characterized by a high oxidative potential (redox potential of +2.07 V vs redox potential of the oxygen molecule O₂ of +1.23 V), lower than only some substances, and significantly higher than that of chlorine (+1.36 V).

It naturally forms in the human body as it is produced by white blood cells called neutrophils; in this way, within our body, it performs the delicate task of counteracting the action of bacteria and mycetes and, in general, of all the abnormal elements present in the body.

In addition to the therapeutic use, ozone can also be used in the cosmetic field, for example for hair care.

In the state of the art, ozone is used during the technical treatments of the hair, such as colouring, bleaching or styling, more specifically during the laying period of the technical treatment or in its progression. Such use usually leads to advantages in terms of durability of the colour or styling, colouring intensity of the hair. In other words, ozone has an adjuvant or performance-enhancing effect of the technical treatment carried out on a user's hair.

In particular, several previous documents show that the use of ozone is functional in terms of performance of the technical treatment, for example in order to accelerate the process of the technical treatment, to make it more efficient or characterized by better results (for example, a greater bleaching effect or a brighter and intense hair colour).

Examples of such applications are described in the following patent documents:
EP0103547 describes an apparatus for the generation of steam and ozone—ozonated steam—in controllable quantities for the treatment of both skin and hair; for the treatment of the hair, a hair dryer (or bell) is used, especially when applying dyes or permings. For example, the treatment with the steam/ozone mixture described in EP0103547 is capable of enhancing the bleaching process of hair keratins, and improving the dyeing and pigmentation processes of the hair. GB820463 concerns a method for the improvement of hair dyeing, bleaching and/or styling processes. A steam and ozone generating apparatus is used in combination with hair treatments. The invention described in GB820463 accelerates and improves the oxidation of the dyes used in hair dyeing; it improves the action of lightening/whitening products; and it is useful in the neutralization of perming lotions.

### Problem of the Prior Art

As already mentioned, the known ozone-based cosmetic methods are intended to enhance the technical treatment by virtue of the oxidising power of O₃.

However, ozone, precisely because of its chemical properties, is harmful to the hair if used improperly: it can lead to structural and functional damages to the hair that are more or less reversible, more specifically damages to the protein components of the hair.

Therefore, the need to develop new techniques for the use of ozone during hair treatments, minimizing the harmful effects of the molecule while exploiting its beneficial and functional properties is felt.

### SUMMARY OF THE INVENTION

The Applicant has identified a method for the cosmetic treatment of the hair that employs ozone. In particular, the method comprising the following steps:
a) conducing a technical treatment on the hair, comprising at least the sub-steps of
   a1) applying on the hair one or more compositions functional to the technical treatment,
   a2) leaving one or more compositions functional to the technical treatment in place on the hair,
   a3) washing the hair to remove the one or more compositions functional to the technical hair treatment from the hair,
b) applying ozone on the hair for a temporal period comprised between 3 to 20 minutes and at a dosage comprised between 180 mg/hr and 400 mg/hr,
wherein
the ozone application step (b) is carried out on damp hair, at the end of the step of conducing of the technical treatment (a).

### Advantages of the Invention

The cosmetic method of the invention is not intended to accentuate or improve the result or the performance of the technical treatment applied on the hair, but focuses on the partial or total regeneration of the primordial structure of the hair before the technical treatment, that is, acting at the level of the disulfide bridges, preferably cysteine disulfide bridges of the hair. This is achieved by using ozone following technical parameters described in the set of claims and in the continuation of the description.

The present invention differs from the common ozone-based cosmetic methods in that:
it envisages applying ozone on the hair at a precise dosage and in a specific time frame. Higher dosages or times of application of the ozone might compromise the hair's constitution. For example, from the embodiment examples, it will be more evident that the use of O₃ over a prolonged temporal period produces structural damages, therefore also functional, to the hair;
it is versatile since it can be exploited for hair care after several technical treatments; moreover, the versatility of use must also be understood from a point of view of the technical equipment available in beauty salons. In fact, the step of applying ozone on the hair (b) can occur through different types of professional tools such as, for example, a hair brush, a hair comb, a hair dryer (with or without diffuser), a hair straightener, a headwash, a hair cap, a hooded dryer, a nebulizer.
it envisages that the step applying the ozone on the hair (b) occurs when the hair is damp. Without being bound by any theory, the Applicant believes that, in the presence of moisture or of a certain percentage of water on the hair or in the vicinity thereof, the contact and the adhesion of the ozone with the proteins of the hair is facilitated, increasing its repair effectiveness. According to the Applicant, ozone is, in fact, able to favour the closure of the cuticles of the hair, increasing the retention of water at the level thereof. This allows to increase the moisture of the hair and make it more elastic, obtaining good results in terms of hydration of the hair and resistance to traction. In addition, the presence of water reduces the concentration of ozone in the air obtaining a less aggressive mixture but still able to oxidise the free sulfhydryl groups SH and restoring the disulfide bonds S—S of the hair proteins;
it allows to achieve good results in terms of hair care since the step of application of ozone on the hair (b) occurs at the end of the technical treatment, in other words after or subsequently to the technical treatment ended; without wishing to be bound by any theory, the Applicant considers that the use of ozone at the end of the technical treatment allows a restructuring and regeneration of the hair;
it allows obtaining good results in terms of hydration, elasticity, resistance to traction/breakage;

The cosmetic method of the invention allows to obtain an economic advantage compared to the treatment with molecular oxygen (O₂). In fact, on the market, the price of oxygen concentrators is higher than that of ozone generators. The former ones require filters and compressors, for example zeolite filters which are notoriously very expensive; on the other hand, for ozone treatment, it is sufficient to have at least one ozone generator. The economic advantage reflects an advantage in terms of simplicity and ease of use of the above-mentioned instrument.

### DESCRIPTION OF THE FIGURES

FIG. 1a—Analysis of the number of free sulfhydryl groups SH on the exposed surface of the hair for the control strand group (“Control”), for the strand group subjected to bleaching (“Bleach.”), for the strand group subjected to bleaching and ozone treatment O₃ (“Bleach. O₃”), for the strand group subjected to bleaching and ozone treatment O₃ for 24 hours (“Bleach. O₃ 24 h”) according to example 1;

FIG. 1b—Analysis of the number of free sulfhydryl groups SH on the exposed surface of the hair for the control strand group (“Control”), for the strand group subjected to perming (“Per”), for the strand group subjected to perming and O₃ ozone treatment (“Per) O₃”, for the strand group subjected to perming and O₃ ozone treatment for 24 hours (“Per O₃ 24 h”) according to example 1;

FIG. 2—Typical FTIR-ATR spectrum of a hair sample;

FIG. 3—Results expressed as weight by grams measured at different measurement times of example 2 relating to dyed control strands;

FIG. 4—Results expressed as weight by grams measured at different measurement times of example 2 relating to ozone dyed strands;

FIG. 5—Results with calculated differences between the weights measured at different times and the initial weight for each strand of example 2 relating to control dyed strands;

FIG. 6—Results with calculated differences between the weights measured at different times and the initial weight for each strand of example 2 relating to ozone dyed strands;

FIG. 7—Results expressed as weight by grams measured at different measurement times of example 2 relating to bleached control strands;

FIG. 8—Results expressed as weight by grams measured at different measurement times of example 2 relating to ozone bleached strands;

FIG. 9—Results with calculated differences between the weights measured at different times and the initial weight for each strand of example 2 relating to control bleached strands;

FIG. 10—Results with calculated differences between the weights measured at different times and the initial weight for each strand of example 2 relating to ozone bleached strands;

FIG. 11—Instrument for measuring the breaking load of example 3;

FIG. 12—Example of a graph obtained from the measurement carried out through the manner of example 3;

FIG. 13—Results of the test of example 3 on bleached strands;

FIG. 14—Results of the test of example 3 on permed strands.

### DETAILED DESCRIPTION OF THE INVENTION

As explained above, O₃ ozone has a better oxidising power than that of O₂ oxygen. Without wishing to be bound by any theory, the Applicant considers that, even more than to a direct action of the ozone, the oxidising capacities of O₃ are attributable to the release of atomic oxygen O, one of the species deriving from ozone decomposition. In particular, atomic oxygen O is obtained by decomposition of the ozone, a highly unstable molecule, in accordance with the following reaction:

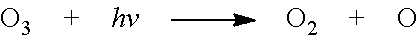

According to the Applicant, ozone favours the formation of disulfide bridges, preferably cysteine disulfide bridges, among the keratin molecules, in order to restructure the hair and make it more resistant, hydrated and luminous; moreover, it is believed that ozone improves the duration of the technical treatments (bleaching, perming, etc.).

For the purposes of the present invention, hair care refers to any process or treatment aimed at restoring or reconstructing, totally or partially, the natural structure of the hair or in any case the one characterizing the hair before the technical treatment.

It is to be noted that the cosmetic method according to the invention based on ozone is not a trichological treatment, where by trichological treatment it is meant a treatment aimed at curing a condition, preferably afflicting the scalp. Examples of trichological treatment are the trichological anti-hair loss treatment (fine, damaged hair and/or hair prone to falling and breaking), the anti-dandruff treatment or the antiseborrhoeic treatment.

By damp hair, for the purposes of the present invention, it is meant both the hair that has just been washed, which has not been dabbed, towel-dried or rubbed; and the hair that has been dabbed, towel-dried or rubbed after washing.

### (a) Conducing a Technical Treatment on the Hair

As previously mentioned, the cosmetic method for hair care comprises the step of conducing a technical treatment on the hair (a), in turn, comprising at least the sub-step of:
a1) applying on the hair one or more compositions functional to the technical treatment
a2) leaving one or more compositions functional to the technical treatment in place on the hair
a3) washing the hair to remove the one or more compositions functional to the technical treatment from the hair.

According to the cosmetic method of the invention, the technical treatment is selected from: hair reconstruction, hair conditioning, colouring, bleaching, perming, and combinations of the foregoing.

A hair reconstruction is a technical treatment aimed at modifying or restoring the natural structure of the hair. It should be noted that the natural shape or structure of the hair is determined by the arrangement of the peptide chains that form the hair, preferably bonds between cysteines, and by the transverse bonds that hold them together. Such bonds are primarily peptide bridges, disulfide, saline, and hydrogen.

Hair conditioning is a technical treatment with the main purpose of detangling the hair, preferably after washing with shampoo. The conditioning products contain surfactants that create a film that, by wrapping the hair, make it slippery and easier to comb.

Colouring is a technical treatment that modifies the natural colour of the hair. Colouring comprises dyes of natural or synthetic origin, of the type, for example, selected from temporary colouring; semi-permanent colouring; permanent colouring.

Bleaching refers to any technical treatment that allows to lighten/bleach the hair by some tone, through a chemical process (destructive oxidation of melanin). Bleached hair usually undergoes an aggressive treatment that modifies the structure thereof, making it thinner and drier.

Bleaching treatments preferably also include treatments to obtain streaks or shatushes. Streaks refer to a technical bleaching treatment that allows to lighten the hair. It is suitable for those with light hair, natural hair and not. It envisages bleaching, over the entire length, of rather regular thin strands.

Shatush refers to a technical treatment that involves a slight cottoning of the strands, then bleaching the slipped tufts: more intense at the tips, barely visible near the roots. In addition, unlike streaks, it is not possible to resume shatush, that is, in order to be able to perform it a second time, it is necessary to cut the bleached lengths.

According to a preferred form of the cosmetic method according to the invention, the technical treatment is selected from bleaching and perming.

“Compositions functional to the technical treatment” means compositions/formulations/products based on natural/semi-natural/synthetic substances used or applied on the hair for each type of technical treatment. For example, for the technical treatment of hair reconstruction, functional compositions can be shampoos, nourishing masks, regenerating and conditioning lotions/mousses/sprays for dry, brittle and damaged hair; for the technical treatment of hair conditioning, functional compositions are conditioners, masks, mineral salts; for the technical treatment of colouring, functional compositions are permanent dyes, gradual or metallic dyes, temporary dyes, semi-permanent dyes, vegetable dyes; for the technical treatment of bleaching, functional compositions are bleaching/lightening formulations, preferably based on oxidising agents, such as hydrogen peroxide, and/or ammonia-based; for the technical treatment of perming, functional compositions are formulations reshaping/waving or styling capacity of the hair structure and/or neutralizing formulations (to avoid excessive structural damage of the hair).

Note that the step of washing the hair to remove the one or more functional composition(s) from the hair provides for removing or permanently eliminating any trace of said composition(s) functional to the technical treatment. Preferably, said washing step is carried out with water.

According to a preferred embodiment, the step of conducing a technical treatment on the hair (a) may comprise further sub-steps (an) which may be prior to, subsequent to, or intermediate to steps (a1)-(a3) of applying, laying, and rinsing the composition functional to the technical treatment. Said further sub-steps (an) are selected from the group consisting of:
applying on the hair curlers or hairpins or other tools suitable for shaping or styling the hair,
applying on the hair a further composition functional to the technical treatment, preferably without rinsing, i.e. without washing with water for the withdrawal/removal of the composition itself from the hair;
applying on the hair a further composition functional to the technical treatment, preferably with rinsing, that is with washing with water for the withdrawal/removal of the composition itself from the hair,
rubbing or towel drying or dabbing the hair, possibly with the help of a towel
and combinations of the foregoing.

### The Technical Treatment is Bleaching

According to a preferred embodiment, the technical treatment is bleaching and the step of conducing a technical treatment on the hair (a) comprises the sub-steps of:
a1′) applying on the hair a first composition functional to the technical treatment, preferably a bleaching/lightening composition,
a2′) leaving the first composition functional to the technical treatment in place on the hair, preferably for a time comprised between 40 and 50 minutes,
a3′) washing the hair to remove the first composition functional to the technical treatment from the hair.

According to an optional embodiment, bleaching comprises other sub-steps subsequent to the hair washing step:
a4′) applying on the hair a second composition functional to the technical treatment, said composition preferably being a toning composition,
a5′) leaving the second composition functional to the technical treatment in place on the hair, preferably for a time comprised between 15 and 30 minutes,
a6′) washing the hair with water to remove the second composition functional to the technical treatment from the hair.

A toning composition is a chemical colouring composition; unlike permanent dyes that act deeper in the hair structure, a toning composition provides that the coloured pigment remains only on the surface of the hair. The toning compositions differ according to the composition and to the intensity of the colouring. Often the toning compositions can dry the hair due to the presence of alcohol therein.

Still preferably, following the hair washing sub-step and before the ozone application step (b), bleaching comprises a further sub-step of dabbing, towel drying or rubbing the damp hair, possibly with the help of a towel.

At the end of the bleaching treatment, and preferably after the sub-step of washing the hair or of dabbing, towel drying or rubbing the damp hair, the cosmetic method of the invention comprises the step of applying ozone on the damp hair (b).

### The Technical Treatment is Perming

According to an alternative embodiment, the technical treatment is perming, and the step of conducing a technical treatment on the hair (a) comprises the sub-steps of:

a0″) selecting one or more strands of hair and wrapping them on curlers,
a1″) applying on the wrapped hair a first composition functional to the technical treatment, said first functional composition being preferably a product for shaping the hair shape (or waving composition),
a2″) leaving the first composition functional to the technical treatment in place on the hair, preferably for a time comprised between 5 and 30 minutes,
a3″) washing the hair to remove the first composition functional to the technical treatment from the hair.

Still preferably, the technical treatment of perming comprises the following further sub-steps:
a4″) applying on the hair a second composition functional to the technical treatment, said second functional composition preferably being a neutralizing composition,
a5″) leaving the second composition functional to the technical treatment in place on the hair,
a6″) removing the curlers or any other equivalent tool from the hair,
a7″) washing the hair to remove the second, optionally the third, composition functional to the technical treatment from the hair.

Still preferably, following the hair washing sub-step and before the ozone application step (b), perming comprises a further step of dabbing, towel drying or rubbing the damp hair.

At the end of the perming treatment, and preferably after the sub-step of washing the hair or of dabbing, towel drying or rubbing the hair, the cosmetic method of the invention comprises the step of applying ozone on the damp hair (b).

### (b) Step of Applying Ozone on the Hair

Preferably, the step of applying ozone on the hair (b) envisages that the ozone is in gaseous form, in mixture with water or in the form of humidified ozone (ozonated water).

For the purposes of the present invention, once ozone is present, the expression “applying ozone on the hair” (b) preferably means
dispensing gaseous ozone directly on the hair; alternatively,
applying a mixture of ozone and water on the hair; alternatively
applying water-solubilized ozone (humidified ozone or ozonated water) on the hair.

For example, ozone in gaseous form can preferably be generated starting from an ozone generator device. An ozone generator preferably comprises at least one fan and two plates, one of which is powered to generate electrical discharges; oxygen conveyed by the fan passes between the plates.

According to a preferred embodiment, the oxygen conveyed between the two plates by the fan is the oxygen present in the atmosphere.

According to an alternative embodiment, the oxygen conveyed between the two plates is pure oxygen, i.e. with a purity ≥98%, such as for example the one contained in an O₂ cylinder or generated by an O₂ concentrator.

The step of applying ozone on the hair (b) is carried out for or in a temporal period comprised between 3 and 20 minutes, preferably comprised between 3 and 15 minutes, preferably between 3 and 10 minutes, preferably between 5 and 10 minutes, even more preferably between 6 and 10 minutes, preferably equal to 6 minutes or alternatively equal to 10 minutes.

The Applicant specifies that, as long as it is within the time parameters according to the ozone application step (b) of the present invention, the restructuring effectiveness of ozone on the hair is obtained; higher timings of use of the ozone risk, however, damaging the hair, with destructuring effects comparable with those resulting from some technical treatments, such as bleaching or perming.

For the purposes of this invention, the ozone application step (b) is carried out on essentially damp hair. By damp hair it is meant
dabbed hair, optionally rubbed or towel-dried, using a cloth or a towel, after washing, in order to remove excess water, thus avoiding the phenomenon of dripping hair when excessively wet; or
wet hair, just after washing.

The other essential condition is that the ozone application step (b) is carried out at the end of the step of conducing the technical treatment (a), i.e. after the last sub-step constituting the technical treatment (a); according to a preferred embodiment, the ozone application step (b) is carried out on the damp hair after abundant rinsing with water to remove any trace of the composition(s) functional to the technical treatment.

The step of applying ozone on the hair (b) preferably occurs by dispensing ozone at a dosage comprised between 180 mg and 400 mg in one hour (h) (extremes included), preferably at a dosage comprised between 200 mg and 300 mg in one hour (h) (extremes included), preferably equal to 200 mg in one hour.

Thus, over a time period preferably comprised between 3 and 20 minutes, the step of applying ozone on the hair (b) provides that the ozone is in an amount preferably comprised between 9 mg and 134 mg, preferably comprised between 9 mg and 120 mg, preferably comprised between 10 and 100 mg, preferably comprised between 10 and 80 mg, preferably comprised between 10 and about 67 mg, preferably comprised between 10 and about 34 mg.

The step of applying ozone the ozone on the hair (b) occurs by means of a handpiece selected from the group consisting of: a hair brush, a hair comb, a hair dryer-preferably with or without diffuser—a hair straightener, a headwash, a hair cap, a hooded dryer, a nebulizer.

The step of applying ozone on the hair (b) preferably includes a dispensing flow comprised between 1 and 5 L/min, more preferably comprised between 1 and 2 L/min.

The dispensing pressure of the ozone in gaseous form is preferably the atmospheric pressure.

### First Ozone Application Mode

According to a preferred embodiment, the step of applying ozone on the hair (b) preferably comprises the following sub-steps:
b1) arranging ozone in gaseous form;
b2) arranging water in liquid form;
b3) dispensing ozone in gaseous form and, at the same time, nebulizing the water in liquid form on the hair.

For the purposes of the present invention, the nebulization of water preferably occurs by means of a nebulizer, i.e. a device that transforms a liquid into an aerosol of very fine droplets; the liquid, introduced by a nozzle, is dispersed by a diffuser.

The step of dispensing the ozone in gaseous form and, at the same time, nebulizing the water in liquid form on the hair (b3) preferably provides that ozone in gaseous form is dispensed on the hair and that, at the same time, the liquid water is nebulized on the damp hair.

Preferably, the step of applying ozone on the hair (b) according to this mode preferably occurs by dispensing ozone in gaseous form at a dosage comprised between 180 mg and 400 mg/h (extremes included), preferably comprised between 180 and 350 mg/h, preferably comprised between 180 and 250 mg/h, preferably comprised between 180 and 200 mg/h, preferably equal to 200 mg/h.

### Second Ozone Application Mode

In a second embodiment alternative to the foregoing, the step of applying ozone on the hair (b) preferably comprises the following sub-steps:
b1′) arranging ozone in gaseous form;
b2′) dispensing ozone in gaseous form on the damp hair.

Note that, according to this second embodiment and unlike the first ozone application mode, the use of water in nebulized liquid form is not envisaged. The moisture already present on the damp hair is instead exploited.

Preferably, the step of applying or dispensing ozone on the hair (b) according to this mode preferably occurs by dispensing ozone in gaseous form at a dosage comprised between 180 mg and 400 mg in one hour (h) (extremes included), preferably comprised between 250 and 400 mg/h, preferably comprised between 350 and 400 mg/h, preferably equal to 400 mg/h.

### Third Ozone Application Mode

A third embodiment of the invention provides that the step of applying ozone on the hair (b) preferably comprises the following sub-steps:
b1″) arranging ozone in gaseous form
b2″) arranging water in liquid form,
b3″) solubilizing the ozone in gaseous form in the water to obtain humidified ozone (or ozonated water), and
b4″) applying the humidified ozone on the damp hair.

Preferably, the step of solubilizing ozone in gaseous form in the water to obtain humidified ozone (or ozonated water) (b3″) includes the use of a bubbler device, known to the person skilled in the art, for the solubilization of ozone in gaseous form in the water. In other words, the gaseous ozone is humidified, through a bubbler, before being applied on the hair.

A further mode of performing the step of ozone solubilization in gaseous form in the water to obtain humidified ozone (or ozone humidification step) (b3″) preferably provides that the ozone in gaseous form is directly mixed or solubilized in water, preferably the water from a sink or headwash. In this way, the gaseous ozone is “immersed” (solubilized) in water before being applied on the hair.

The passage of the ozone stream in water preferably makes it possible to dissolve the gas, so that the micro-drops of humidified ozone (or ozonated water) adhere more effectively to the hair, improving its effectiveness. For the purposes of the present invention, preferably the bubbler has a volume ≥350 ml and ≤500 ml, preferably equal to 500 ml.

For the purposes of this invention, “applying humidified ozone on the damp hair” (b4″) means applying the humidified or water-solubilized ozone (humidified ozone or ozonated water) on the damp hair.

Preferably, the step of applying the humidified ozone (or ozonated water) on the hair (b4″) occurs by nebulizing (“vaporizing”) the humidified ozone, i.e. by means of a nebulizer or vaporizer; according to another mode, the step of applying the humidified ozone on the hair (b4″) preferably occurs by pouring (or outflow of) at least a part of the humidified ozone (or ozonated water) mentioned above on the already damp hair of a user.

According to an alternative embodiment, the method may provide for the use of a catalyst that contributes to improving the effectiveness of the treatment, by recovering chemical species produced in the ozone application phase and which, if not recovered, would be lost to the environment.

Preferably, the step of applying ozone on the hair (b) according to the third mode preferably occurs by applying ozone at a dosage comprised between 180 mg and 400 mg in one hour (h) (extremes included), preferably comprised between 200 and 400 mg/h, preferably comprised between 200 and 350 mg/h, preferably comprised between 200 and 250 mg/h, preferably equal to 200 mg/h.

According to the third ozone application mode, considering the solubility of the ozone in gaseous form in water at a temperature preferably comprised between 2° and 30° C., the ozone content (or concentration) in the humidified ozone (or ozonated water) is preferably comprised between 350 mg and 600 mg, preferably comprised between 400 mg and 570 mg per litre of humidified ozone (or ozonated water).

Preferably, after step of applying ozone on the hair (b), respecting the timings for the purposes of the invention, the hair can be dried, preferably by means of a common hair dryer.

### EXAMPLES

Below the Applicant reports examples of the invention, for illustrative and non-limiting purposes only.

It should be noted that the term “Bleach.” refers to the bleaching treatment, whereas the term “Per” refers to the perming treatment.

### Example 1—Effect of the Ozone Treatment According to the Cosmetic Method of the Invention on Bleached and Permed Hair

The purpose of the study is to characterize from a chemical point of view the effect of the treatment with ozonated air on the hair after bleaching treatments with aqueous hydrogen peroxide 30 volumes (9% by weight) or after perming.

The study was conducted on standard bleached hair samples of 8 g each treated as described below:

TABLE 1
Treatments carried out on the samples

| No. | | |
|---|---|---|
| Samples | Initials | Treatment |
| 1 | Control | No treatment |
| 2 | Bleach. | Bleaching with H₂O₂ 30 Vol |
| 3 | Bleach. | Bleaching with H₂O₂ 30 Vol + treatment with |
| | O3 | ozonated air (400 mg/h) bubbled in water for 10 |
| | | minutes on wet hair |
| 4 | Bleach. | Bleaching with H₂O₂ 30 Vol + treatment with |
| | O3 24 h | ozonated air (400 mg/h) directed for 24 hours |
| | | on wet hair |
| 5 | Per | Perming |
| 6 | Per O3 | Perming + treatment with ozonated air (400 mg/h) |
| | | bubbled in water for 10 minutes on wet hair |
| 7 | Per O3 | Perming + treatment with ozonated air (400 mg/h) |
| | 24 | directed for 24 hours on wet hair |

In the case of ozone treatment according to the method of the invention, ozonated air produced by an ozonator with a flow rate of 400 mg of ozone per hour is used. It is believed, without being bound by any theory, that ozonated air is more effective if it is bubbled in water before application on the hair because the presence of moisture favours the contact of ozone with the proteins of the hair; moreover, the presence of water reduces the concentration of ozone in the air obtaining a less aggressive mixture but still able to oxidise the sulfhydryl groups restoring the disulfide bonds.

Two types of analysis were carried out on the samples:
a. determination of the free sulfhydryl groups by Vis-UV spectrophotometric method using dithiobis-(4-pyridine) (DTBP) as SH group sensitive reagent (Winther J R, Thorpe C. Biochim Biochim Acta, 2014, Biophys 1840, doi: 10.1016/j.bbagen.2013.03.031; RienerCK, Kada G, Gruber HJ. Anal Bioanal Chem, 2002, 373, 266-276 doi: 10.1007/s00216-002-1347-2; Hasen R E, Ostergarrd H, Norgaard P, Winther J R. Anal Biochem, 2007, 363, 77-87);
b. determination of the degradation index of keratin by reflectance Fourier transform infrared spectroscopy (ATR-FTIR) (Messrs V, Lewis DM. International Journal of Cosmetic Sciences, 1996, 19, 1-13).
   a. Determination of the Free Sulphhydryl Groups

The formation of disulfide (S—S) bonds in hair keratin is an important factor determining its structure and stability. Bleaching or perming treatments alter the number of disulfide bonds both because they reduce them to SH sulfhydryl groups and because they oxidatively degrade cysteine and its cystine dimer. The analysis of the free sulfhydryl groups SH is an index of good health of the hair; in fact, if these groups are present in a too large number it means that the disulfide bonds have been broken and keratin loses stability. The number of the sulfhydryl groups can be determined through the use of dithiobis-(4-pyridine) which, by reacting with the SH groups, forms a compound that absorbs ultraviolet light at 324 nm and allows the quantitative determination thereof through Vis-UV spectrophotometry.

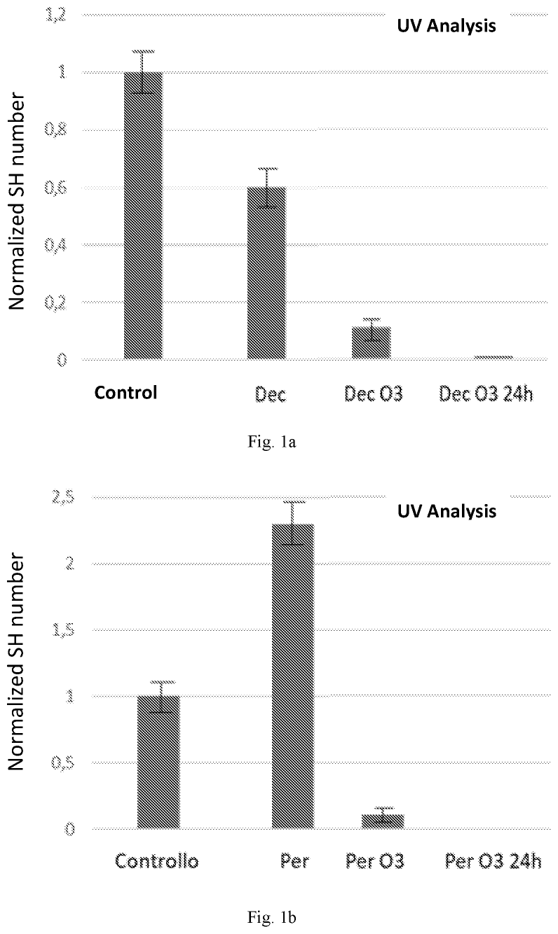

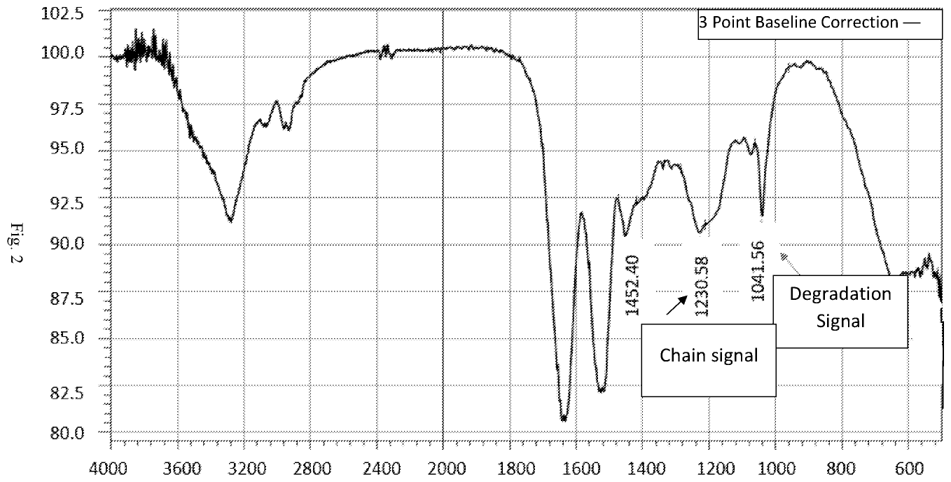

As can be seen in FIG. 1a and in FIG. 1b compared to standard hair, the bleaching treatment reduces the amount of free SH groups, while perming increases it. This is due to the fact that the bleaching treatment, being strongly oxidising, degrades the surface cysteine of the hair reducing the sensitivity to the reagent. In particular, the Applicant considers that the phenomenon of reduction of the SH groups following bleaching (FIG. 1a) should not be understood as connected to the formation of S—S disulfide bridges, but linked to the constitution of other bonds, therefore different from disulfide bonds, that the SH thiol group can form with other molecular residues.

In any case, the subsequent ozone treatment, according to the cosmetic method of the invention, further reduces the amount of free SH sulfhydryl groups; this represents an indirect measurement of the formation of the disulfide bridges. However, the doubt remains that a further oxidative treatment can also lead to the degradation of keratin in cysteine, which would in any case decrease the content of sulfhydryl groups, an aspect that is clarified by the study in the next paragraph in FTIR-ATR spectroscopy.

b. Determination of Keratin Degradation

Infrared spectroscopy represents a valid and fast method for the determination of protein degradation as it highlights the structural characteristics of the molecules, such as the presence of new functional groups deriving from oxidative processes. In particular, it is possible to determine an index of keratin damage by monitoring the signals related to the protein chain (i.e. the signal at 1230-1250 cm⁻¹ related to amide bonds) and the signals related to the oxidation of cystine and cysteine (1040 cm⁻¹). The degradation index can be calculated by normalizing to 100 the signal area at 1230-1250 cm⁻¹ and by calculating the signal area at 1040 cm⁻¹, accordingly. The higher this value, the more degraded the cysteine (FIG. 2).

Table 2 shows the results of the analyses carried out.

TABLE 2

| | Degradation | | Degradation |
|---|---|---|---|
| Sample | index | Sample | index |
| Control | 1.0 (2) | Control | 1.0 (2) |
| Bleaching (Bleach.) | 3.1 (3) | Perming (Per) | 2.6 (2) |
| Bleach. O₃ | 1.4 (1) | Per O₃ | 1.3 (1) |
| Bleach. O₃ 24 h | 2.2 (1) | Per O₃ 24 h | 2.7 (2) |

Note that after bleaching or perming the hair is more damaged than in the initial situation, and that the ozone treatment according to the cosmetic method of the invention for 10 minutes improves the situation thereof. This could appear to be in contrast to the oxidising power of ozone, which should not lead to a restoration of the oxidised cysteine. However, the reformation of disulfide bridges, which close the cuticles, causes a morphological change of the hair, exposing in a lesser way the damaged parts that remain inside the cuticles themselves. This undoubtedly also improves the aesthetic appearance of the hair. Prolonged treatment with ozone worsens the situation demonstrating that the ozone itself, being in any case an aggressive oxidant, in the long run triggers oxidative processes that lead to further degradation of cysteine.

### 1.1 Conclusions

Chemical investigations based on spectroscopic methods confirm that the treatment with ozonated air bubbled in water for 10 minutes according to the invention improves the chemical structure of the hair by restoring the disulfide bonds of the cysteines that have been damaged by the previous treatments.

### Example 2—Moisture Absorption Test

The purpose of the study is to quantify the hydration level of the hair obtained after the cosmetic method of the invention in comparison to the hydration level of untreated hair.

### 2.1. Materials and Methods

Ozone device: able to dispense ozone; the hair is inserted into plastic bags into which ozone is blown.

Hair braids: for the execution of the test, two different types of hair are distinguished:
dyed strands, derived from “Italian Virgin Hair” hair braids, about 16 cm long, purchased from the company Imhair—Palermo. They are composed of auburn-coloured straight hair, subsequently dyed by permanent colouring;
bleached strands, derived from “Gold” depigmented hair braids, about 16 cm long, purchased from the company Imhair-Palermo. They are composed of bleached, blond-coloured straight hair.

Basic shampoo: all the strands used, both control and treated, have been previously washed with a basic shampoo, in order to equalize the surface. The qualitative-quantitative formula of the shampoo is shown in table 3.

TABLE 3

| INCI | % w/w |
|---|---|
| AQUA | 81.50 |
| SODIUM LAURETH SULFATE | 13.20 |
| COCAMIDE DEA | 2.00 |
| AQUA, SODIUM | 0.80 |
| HYDROXYMETHYLGLICYNATE | |
| SODIUM CHLORIDE | 2.50 |
| | 100.00 |

Permanent colouring: COLOR+dyeing with colouring 5.60 (light auburn, intense red), activated by oxidising cream in a 1:2 ratio, allowed for 40′ in place.

Measuring instrument: Mettler Toledo analytical scale (mod. AB104-S/FACT) maximum flow rate of 110 g and sensitivity up to 0.1 mg.

### 2.2 Measurement Method

The study involves the execution of the test on two different types of hair:
hair dyed by permanent colouring: for this type, the comparison between the group of control strands and the group of strands treated for 6 minutes by means of ozone is envisaged;
bleached hair: for this type, the comparison between the group of control strands and the group of strands treated for 6 minutes by means of ozone is envisaged.

Each group consists of 6 strands of similar weight, held together by elastic cotton. All strands were washed using the basic shampoo of Table 3 for about 2 minutes.

The ozone treatment according to the cosmetic method of the invention was performed on wet strands in both cases for 6 minutes.

All the strands were dried with a common hair dryer (2000 watts) for 10 minutes, at maximum speed and intermediate temperature.

After drying, each strand was weighed by analytical scale (TO).

Each strand was kept for 24 hours at a constant temperature of 30° C. and 70% relative humidity. The strands were weighed at regular time intervals: after 1 hour (T1), 2 hours (T2) and 24 hours (Tf), to check the hydration level reached.

Subsequently, the strands were removed from the thermostat chamber and brought back to room temperature (about 21° C., 50% RH). They were weighed again at regular intervals, after 1 hour (T3), 2 hours (T4) and 24 hours (Tfg) after removal.

### 2.3 Results

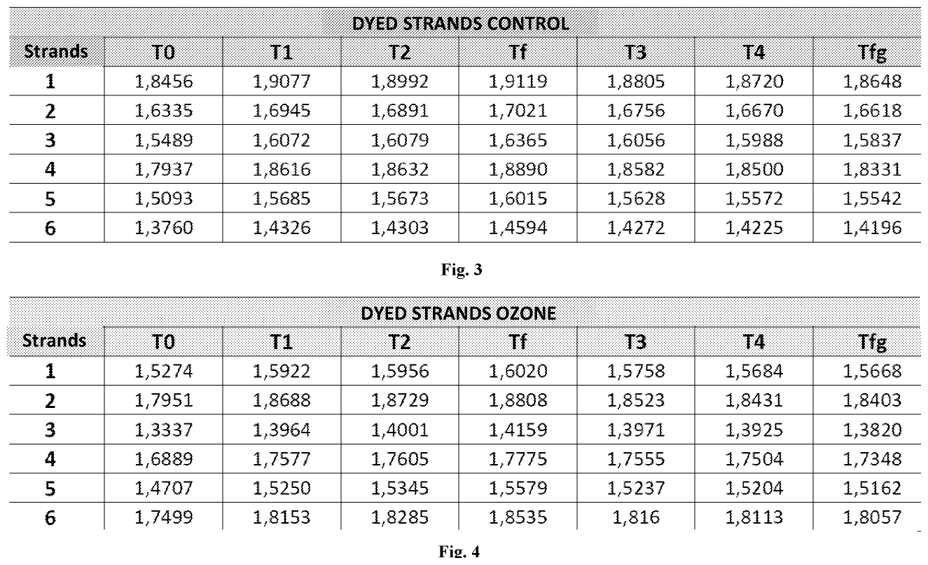

FIGS. 3-6 and 7-10 show the weights in grams measured at the different measurement times: immediately after treatment (TO), after 1 hour (T1), 2 hours (T2) and 24 hours (Tf) in a thermostated chamber, and after 1 hour (T3), 2 hours (T4) and 24 hours (Tfg) at room temperature.

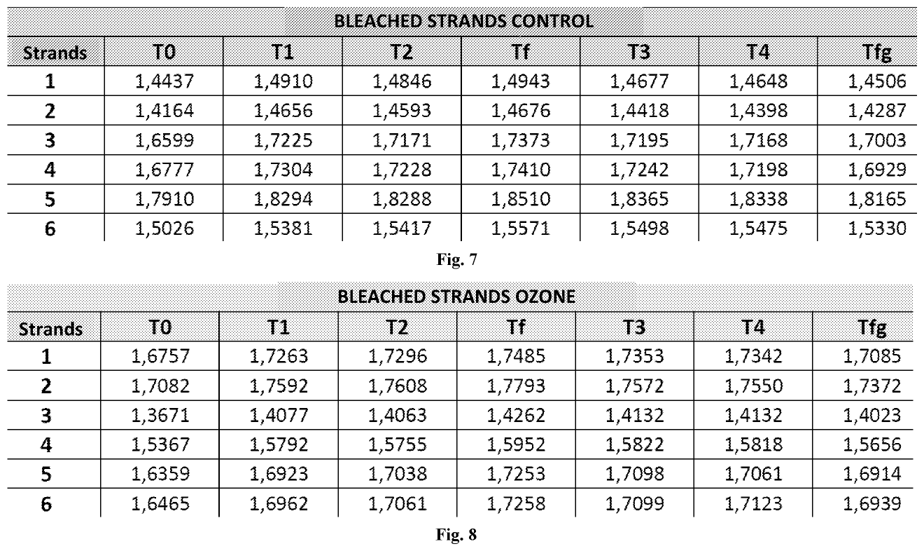

The results related to the dyed strands are shown in FIGS. 3-6, while those related to the bleached strands are shown in FIGS. 7-10.

### 2.3.1 Dyed Strands

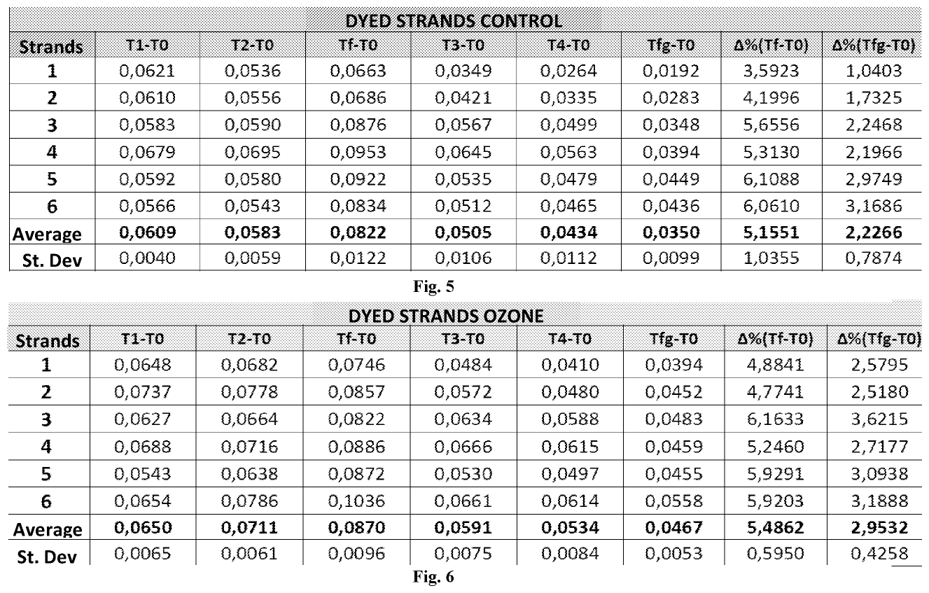

In particular, FIGS. 5 and 6 show tables with the calculated differences between the weights measured at the different times and the initial weight for each strand.

Applying the t-test function to evaluate whether the weight variation measured between the final time (Tfg) and the initial time (TO) is statistically significant, we compare the control group and the group of strands treated for 6 minutes by means of ozone. We also compare the calculated % variations between final time and initial time. When the p-value≤0.05, the difference between the values is considered statistically significant.

TABLE 4

| | t-test Tfg − T0 |
|---|---|
| CONTROL vs OZONE dyed strands | 0.0102 significant |

| | t-test Δ%(Tfg − T0) |
|---|---|
| CONTROL vs OZONE dyed strands | 0.0372 significant |

### 2.3.2 Bleached Strands

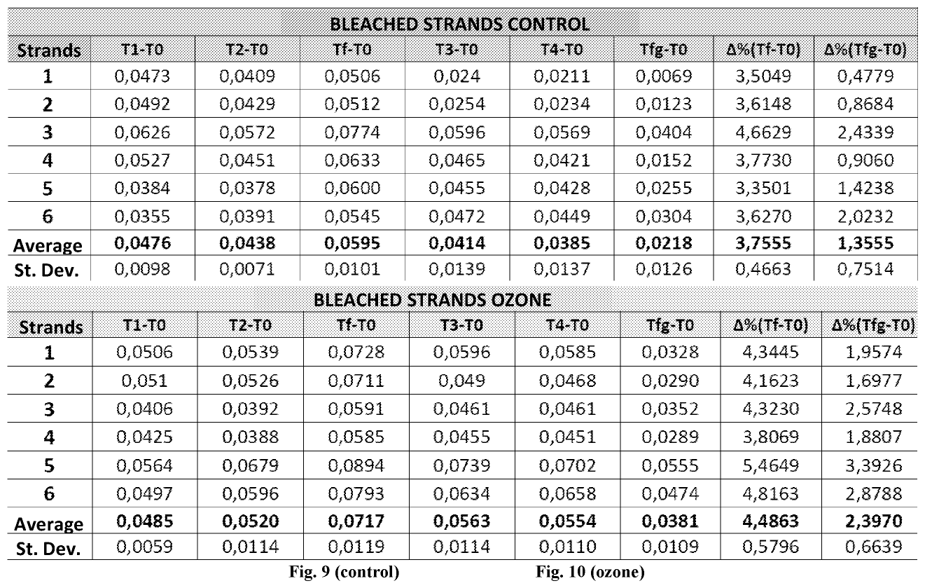

FIGS. 9 and 10 show tables with the calculated differences between the weights measured at the different times and the initial weight for each strand.

We apply the t-test function to evaluate whether the weight variation measured between the final time (Tfg) and the initial time (TO) is statistically significant, we compare the control group and the group of strands treated for 6 minutes by means of ozone. We also compare the calculated % variations between final time and initial time. When p≤0.05, the difference between the values is considered statistically significant.

TABLE 5

| | t-test Tfg − T0 |
|---|---|
| CONTROL vs OZONE bleached strands | 0.0222 significant |
| | t-test Δ%(Tfg − T0) |
| CONTROL vs OZONE bleached strands | 0.0095 significant |

### 2.4 Conclusions

### 2.4.1 Dyed Strands

The ozone-treated dyed strands have a significantly greater variation in weight between the initial and final time than the control strands; in fact, by evaluating the trend, a greater amount of adsorbed humidity, especially between T1 and T2, and the maintenance of a higher level of hydration until the final time can be observed.

### 2.4.2 Bleached Strands

The ozone-treated bleached strands have a significantly greater variation in weight between the initial and final time than the control strands. By observing the trend, in fact, the ozone-treated strands, especially between T1 and T2, have adsorbed a greater amount of moisture since the beginning. Both groups of treated bleached strands have maintained a higher hydration level until the final time.

### Example 3—Hair Repair Power Test

The purpose of the study is to evaluate the load that must be applied to hair to reach the breakage thereof, when subjected to constant traction force. The test was carried out in comparison, on ozone-treated hair and untreated hair.

### 3.1. Materials and Methods

Ozone device: able to dispense ozone; the hair is inserted into plastic bags into which ozone is blown.

Hair braids: for the execution of the test, two different types of hair are distinguished:
bleached strands, derived from “Gold” depigmented hair braids, about 16 cm long, purchased from the company Imhair-Palermo. They are composed of bleached, blond-coloured straight hair;
permed strands, derived from “Italian Virgin Hair” hair braids, about 16 cm long, purchased from the company Imhair-Palermo. They are composed of auburn-coloured straight hair, on which a perming treatment has been carried out.

Basic shampoo: all the strands used, both control and treated, have been previously washed with a basic shampoo, in order to equalize the surface. The qualitative-quantitative formula of the shampoo is shown in table 6.

TABLE 6

| INCI | % w/w |
|---|---|
| AQUA | 81.50 |
| SODIUM LAURETH SULFATE | 13.20 |
| COCAMIDE DEA | 2.00 |
| AQUA, SODIUM | 0.80 |
| HYDROXYMETHYLGLICYNATE | |
| SODIUM CHLORIDE | 2.50 |
| | 100.00 |

Perming products: a curling lotion and a neutralizing agent (Inebrya), applied on strands of hair fastened on curlers, were used for perming.

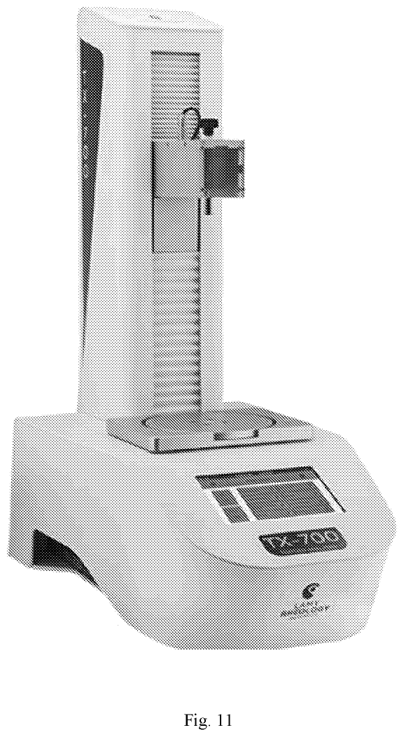

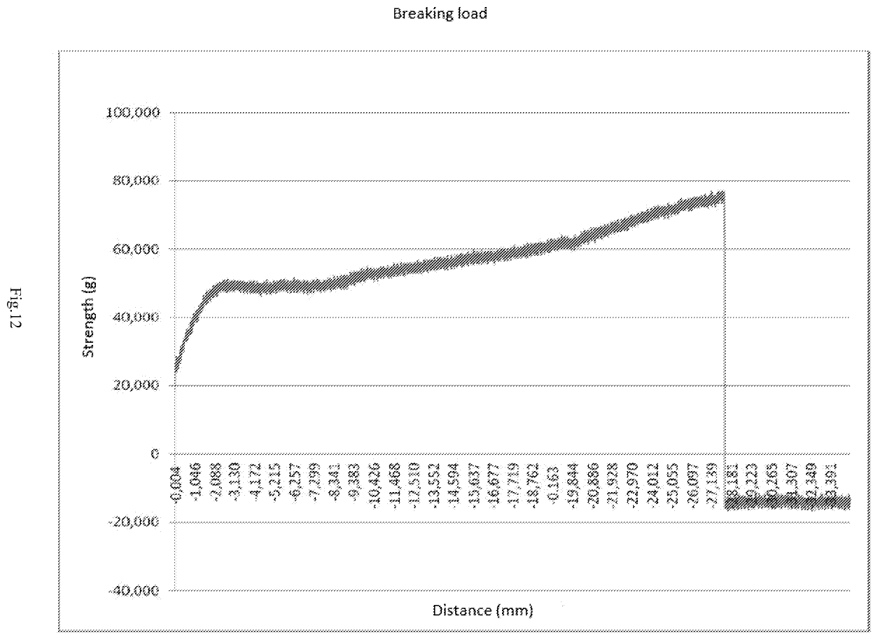

Measuring instrument: the Texture Analyzer TX-700 by Lamy Rheology (FIG. 11), equipped with an accessory “Noodle Tensile Fixture” to which the hair is fastened, was used to measure the breaking load.

The setting parameters used for the traction test are as follows: speed 1 mm/s, distance 40 mm, trigger force 10.2 g.

The instrument provides the recorded force values (g) as a function of the distance (mm). It is possible to extrapolate the maximum force data measured, to which the hair suffers breakage. An example of a graph (FIG. 12) obtained from the measurement carried out through the described mode is shown.

### 3.2 Preparation of the Strands

A hair braid, for both types, was previously washed using a basic shampoo of table 6 for about 2 minutes. This procedure is carried out in order to equalize the conditions of the hair. From each braid, 6 strands weighing about 1-2 g were obtained, held together by elastic cotton. The strands are divided as follows: 2 strands per control group; 2 strands per ozone-treated group.

### 3.2.1 Bleached Strands

After washing with basic shampoo, the control strands were dried for 10 minutes by means of a common hair dryer (2000 Watt), at maximum speed and at intermediate temperature. The other strands were treated in a wet condition with ozone for 6 minutes and then dried for 10 minutes.

### 3.2.2 Permed Strands

After washing with basic shampoo, the strands were wrapped on curlers and the perming treatment was carried out. After being rinsed, the control strands were dried for 10 minutes by means of a common hair dryer (2000 Watt), at maximum speed and intermediate temperature. The other strands were treated in a wet condition with ozone for 6 minutes and then dried for 10 minutes.

### 3.3 Results

The results of the measurements carried out on each group of strands are shown below. Mean value and standard deviation were calculated on the values of the breaking load in grams. They were compared by t-test function for the group of control vs ozone strands. The difference between the values of the series is considered statistically significant with a probability value p≤0.05.

### 3.3.1 Bleached Strands

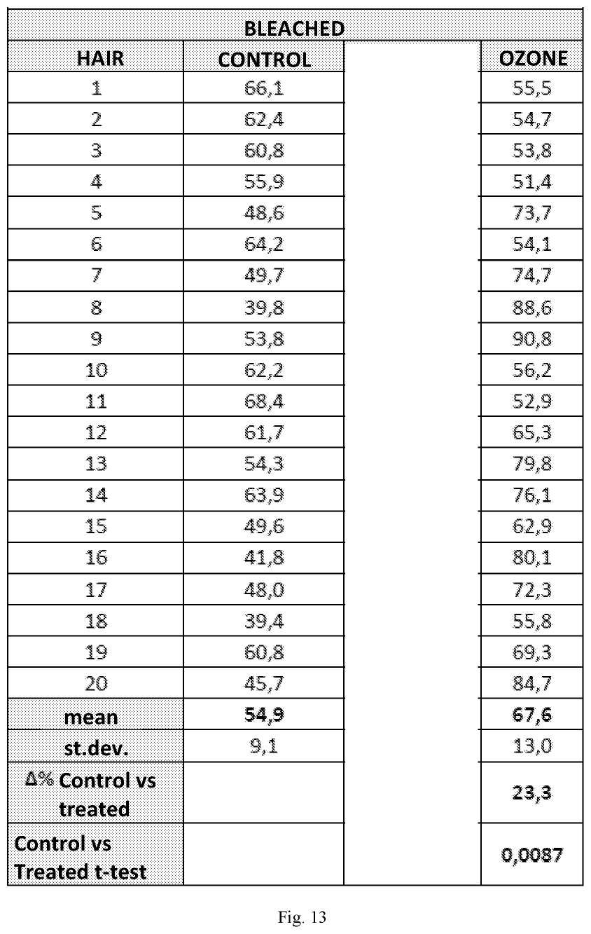

The results are shown in FIG. 13.

From the statistical comparison we obtain:

TABLE 7

| | |
|---|---|
| BLEACHED CONTROL VS BLEACHED OZONE | p < 0.05 |

### 3.3.2 Permed Strands

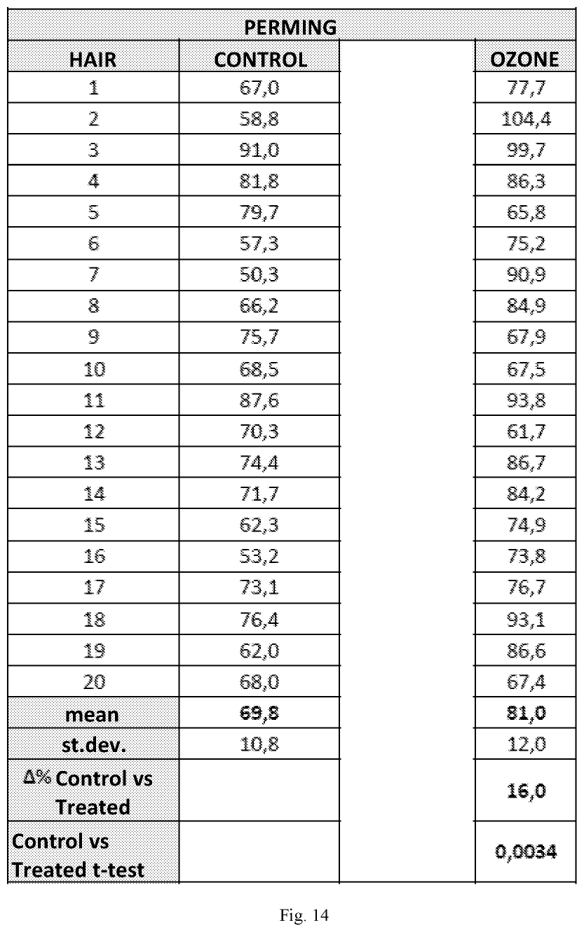

The results are shown in FIG. 14.

From the statistical comparison we obtain:

TABLE 8

| | |
|---|---|
| PERMED CONTROL VS OZONE BLEACHED | p < 0.05 |

### 3.4 Conclusions

The ozone treatment leads, both on bleached and permed strands, to a significant improvement in terms of resistance to the breaking load. The increase is greater after the ozone treatment.

The invention claimed is:

1. Cosmetic method for hair care comprising the following steps:
a) conducing a technical treatment on the hair, comprising at least the sub-steps of
a1) applying on the hair one or more compositions functional to a technical treatment,
a2) leaving one or more compositions functional to the technical treatment in place on the hair,
a3) washing the hair to remove from the hair the one or more compositions functional to the technical treatment,
b) applying ozone on the hair for a temporal period comprised between 3 to 20 minutes and at a dosage comprised between 180 mg/hr and 400 mg/hr,
wherein
the ozone application step (b) is carried out on damp hair, at the end of the step of conducing of the technical treatment (a).

2. Cosmetic method according to claim 1, wherein the technical treatment is selected from the group consisting of: hair reconstruction, hair conditioning, colouring, bleaching, perming, and combinations thereof.

3. Cosmetic method according to claim 1, wherein the technical treatment is selected from bleaching and perming.

4. Cosmetic method according to claim 1, wherein the technical treatment is bleaching and wherein the step of conducing a technical treatment on the hair (a) comprises at least the sub-steps of:
applying on the hair a first composition functional to the technical treatment, said functional composition being a bleaching composition,
leaving the bleaching composition in place on the hair,
washing the hair to remove the bleaching composition from the hair.

5. Cosmetic method according to claim 1, wherein the technical treatment is perming and wherein the step of conducing a technical treatment on the hair (a) comprises at least the sub-steps of:
selecting one or more strands of hair and wrapping them on curlers,
applying on the hair a first composition functional to the technical treatment, said first functional composition being a waving composition,
leaving the waving composition on the hair,
washing the hair to remove the waving composition,
applying on the hair a second composition functional to the technical treatment, said second functional composition being a neutralizing composition,
leaving the neutralizing composition on the hair,
removing the curlers and washing the hair to remove the neutralizing composition.

6. Cosmetic method according to claim 1, wherein the step of applying the ozone on the hair (b) comprises the following sub-steps:
b1) arranging ozone in gaseous form;
b2) arranging water in liquid form;
b3) dispensing ozone in gaseous form on the hair and, at the same time, nebulizing the water in liquid form on the damp hair;
or
b1′) arranging ozone in gaseous form;
b2′) dispensing ozone in gaseous form on the damp hair.

7. Cosmetic method according to claim 1, wherein the step of applying the ozone on the hair (b) comprises the following sub-steps:
b1″) arranging ozone in gaseous form
b2″) arranging water in liquid form,
b3″) solubilizing the ozone in gaseous form in the water to obtain humidified ozone, and
b4″) applying the humidified ozone on the damp hair.

8. Cosmetic method according to claim 7, wherein the ozone content in the humidified ozone is comprised between 350 mg and 600 mg per liter of humidified ozone.

9. Cosmetic method according to claim 1, wherein the step of applying the ozone on the hair (b) occurs by a means selected from the group consisting of: a hair brush, a hair comb, a hair dryer (with or without diffuser), a hair straightener, a headwash, a hair cap, a hooded dryer, a nebulizer.

10. Cosmetic method according to claim 1, wherein the step of applying ozone on the hair (b) includes a dispensing flow comprised between 1 and 5 L/min.
